(19) United States
(12) Patent Application Publication
(10) Pub. No.: US 2010/0330556 A1
(43) Pub. Date: Dec. 30, 2010

(54) GENOME ANALYSIS USING A NICKING ENDONUCLEASE

(76) Inventors: Brian Jon Peter, Los Altos, CA (US); Amir Ben-Dor, Kfar Kava (IL); Zohar Yakhini, Ramat HaSharon (IL); Holly Hogrefe, San Diego, CA (US)

Correspondence Address: Agilent Technologies, Inc. in care of:;CPA Global, P. O. Box 52050, Minneapolis, MN, 55402, US

(21) Appl. No.: 12/495,199

(22) Filed: Jun. 30, 2009

(51) Int. Cl. C12Q 1/68

(52) U.S. Cl. 435/6

(57) ABSTRACT

A method of genome analysis is provided. In certain embodiments, the method of comprises: a) contacting a genomic sample comprising a double-stranded DNA with a site-specific nicking endonuclease to provide a nicked double-stranded DNA comprising a plurality of nick sites, in which the nicking endonuclease nicks a site adjacent to a variable nucleotide; b) contacting the nicked double-stranded DNA with a polymerase in the presence of a nucleotide composition comprising a first labeled nucleotide comprising a first label, thereby producing a labeled double-stranded DNA that is not labeled at every nick site; c) stretching out the labeled double-stranded DNA to provide a stretched, labeled double-stranded DNA; and d) imaging the stretched, labeled double-stranded DNA to identify a labeling pattern on the stretched labeled double-stranded DNA.

Microarray and sequencing technologies provide high-resolution measurements of DNA, and traditional cytogenetics methods such as (e.g., FISH and karyotyping) provide a chromosome-wide view. Optical mapping techniques also enable measurement of sequence features of chromosome-sized DNA fragments. However, these mapping techniques are powerful when used with a sequence-specific labeling technique that can label double-stranded DNA, leaving the target DNA intact. Site-specific nicking endonucleases create a single-stranded DNA break at restriction enzyme recognition sequences in the DNA. Nicking endonuclease digestion can be used to target nick-translation reactions on DNA, and this method can be used to incorporate labels at the recognition sites of the nicking endonucleases. Thus, nicking endonuclease digestion combined with nick translation in the presence of labeled nucleotides can be used to incorporate labels at specific distances that depend on the underlying sequence.

However, problems remain that limit a prevalent adoption of this genome decoration technique. In particular, techniques for genome decoration need to be optimized, and assays designed to exploit the freedom of parameter and method choices. As such, there remains need for measurement technologies to provide some sequence and mapping information on a scale of about 10 to about 1000 kilobases.

This disclosure relates in part to a method of genome analysis using a site specific nicking endonuclease and to the design of specific embodiments of said method.

### SUMMARY

A method of genome analysis is provided. In certain embodiments, the method comprises: a) contacting a genomic sample comprising a double-stranded DNA with a site-specific nicking endonuclease to provide a nicked double-stranded DNA comprising a plurality of nick sites, in which the nicking endonuclease nicks a site adjacent to a variable nucleotide; b) contacting the nicked double-stranded DNA with a polymerase in the presence of a nucleotide composition comprising a first labeled nucleotide comprising a first label, thereby producing a labeled double-stranded DNA that is not labeled at every nick site; c) stretching out the labeled double-stranded DNA to provide a stretched, labeled double-stranded DNA; and d) imaging the stretched, labeled double-stranded DNA to identify a labeling pattern on the stretched labeled double-stranded DNA.

### BRIEF DESCRIPTION OF THE FIGURES

FIG. 1 schematically illustrates an embodiment of the method described herein.

FIG. 2 schematically illustrates certain features of some embodiments of the method described herein.

FIG. 3 schematically illustrates certain features of another embodiment of the method described herein.

### DEFINITIONS

The term “sample”, as used herein, relates to a material or mixture of materials, typically, although not necessarily, in liquid form, containing one or more analytes of interest.

The term “genome”, as used herein, relates to a material or mixture of materials, containing genetic material from an organism. The term “genomic DNA” as used herein refers to deoxyribonucleic acids that are obtained from an organism. The terms “genome” and “genomic DNA” encompass genetic material that may have undergone amplification, purification, or fragmentation. The term “test genome,” as used herein refers to genomic DNA that is of interest in a study. The test genome may encompass the entirety of the genetic material from an organism, or it may encompass only a selected fraction thereof: for example, the test genome may encompass one chromosome from an organism with a plurality of chromosomes.

The term “reference genome”, as used herein, refers to a sample comprising genomic DNA to which a test sample may be compared. In certain cases, reference genome contains regions of known sequence information.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes. Nucleotides may include those that when incorporated into an extending strand of a nucleic acid enables continued extension (non-chain terminating nucleotides) and those that prevent subsequent extension (e.g. chain terminators).

The term “chain terminator” or “chain terminator nucleotide”, as used herein, denotes a nucleotide as defined above but with certain modifications to prevent nucleic acid extension from the chain terminator nucleotide. Stated differently, a chain terminator is derived from a monomeric unit of nucleic acid polymers but is modified such that they prevent subsequent polymerization. One example of a chain terminator is dideoxynucleotide. Another example of a chain terminator is an acyclonucleotide. Chain terminators may comprise a fluorescent or other detectable label (referred to as “dye terminators”) or may be unlabeled.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide”, as used herein, denotes a single-stranded multimer of nucleotides from about 2 to 500 nucleotides, e.g., 2 to 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200, up to 500 or more nucleotides in length, for example.

The term “duplex” or “double-stranded” as used herein refers to nucleic acids formed by hybridization of two single strands of nucleic acids containing complementary sequences. In most cases, genomic DNA are double-stranded.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “chromosomal region” or “chromosomal segment”, as used herein, denotes a contiguous length of nucleotides in a genome of an organism. A chromosomal region may be in the range of 1000 nucleotides in length to an entire chromosome, e.g., 100 kb to 10 MB for example.

The term “sequence alteration”, as used herein, refers to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect a difference, e.g. abnormality, in chromosome structure, such as an inversion, a deletion, an insertion or a translocation relative to a reference chromosome, for example.

As used herein, the term “endonuclease” refers to a family of enzymes that has an activity described as EC 3.1.21, EC 3.1.22, or EC 3.1.25, according to the IUBMB enzyme nomenclature. Site-specific endonucleases recognize specific nucleotide sequences in double-stranded DNA. Some sequence-specific endonucleases cleave only one of the strands in a duplex and are referred to herein as “nicking endonucleases”. Nicking endonuclease catalyzes the hydrolysis of a phosphodiester bond, resulting in either a 5′ or 3′ phosphomonoester.

A “site-specific nicking endonuclease”, as used herein, denotes a nicking endonuclease that cleaves one strand of a double-stranded nucleic acid by recognizing a specific sequence on the nucleic acid. The cleavage site or “nick site” of the phosphodiester backbone may fall within or immediately adjacent the recognition sequence of the site-specific nicking endonuclease.

As used herein, the term “variable nucleotide” in the context of a nick site for a site-specific nicking endonuclease, denotes a nucleotide immediately 3′ or 5′ to a nick site that may be variable from nucleic acid to nucleic acid. In other words, if a site-specific nicking endonuclease nicks a site adjacent to a variable nucleotide, the resultant nick sites contain XA/Xv or AX/vX where A and v represent the nick site on the same strand or opposite strand, respectively, and X is A, T, G, or C. For example, Nb.BsrDI, Nb.BtsI, Nt.AlwI, Nt.BspQI, and Nt.BstNBI nick a site adjacent to a variable nucleotide because they nick at the following sites: GCAATGvX, GCAGTGvX, GGATCNNNNAX, GCTCTTCXAX, GAGTCNNNXAX, respectively. Nb.BbvCI, Nb.BsmI, and Nt.BbvCI do not nick adjacent to a variable nucleotide because they nick at the following sites: CCTCAvGC, GAATGvC, and CCATCAGC, respectively, and nucleotides adjacent to their nick sites are always the same from one nucleic acid sample to another.

As used herein, the term “data” refers to refers to a collection of organized information, generally derived from results of experiments in lab or in silico, other data available to one of skilled in the art, or a set of premises. Data may be in the form of numbers, words, annotations, or images, as measurements or observations of a set of variables. Data can be stored in various forms of electronic media as well as obtained from auxiliary databases.

The term “stretching”, as used herein, refers to the act of elongating a DNA molecule so to minimize the amount of tertiary structures, e.g. unfolding coiled DNA structures.

The term “homozygous” denotes a genetic condition in which identical alleles reside at the same loci on homologous chromosomes. In contrast, “heterozygous” denotes a genetic condition in which different alleles reside at the same loci on homologous chromosomes.

“Color”, as used herein, refers to the wavelength at which the emission spectrum of a label reaches a maximum. For example, a label that is referred herein as red has an emission spectrum with a maximum at about 650 nm.

The term “imaging” refers not only to the collection of data in visible wavelengths (e.g., light microscopy), but also to the collection of wavelengths not visible to the naked eye, e.g., infrared or ultraviolet wavelengths, or the collection of electrons, e.g., electron microscopy. Furthermore, imaging may refer to the collection of data in a form other than light, e.g., surface topography measurements collected by atomic force microscopy, which are then rendered as an image with the aid of a computer. Data collection systems suitable for imaging may include light microscopes, atomic force microscopes, transmission electron microscopes, scanning tunneling microscopes, near-field detection systems, total internal reflection microscopes, and the like.

As used herein, the term “labeling pattern” refers to a pattern of labels that is generated in an image when labeled nucleotides incorporated into a stretched double-stranded nucleic acid are visualized. The labeling pattern in an image is derived from wavelengths of the spectrum peak emitted by the labels (e.g. colors). A labeling pattern consists of the order of the observed labels and/or of spatial components (e.g. distance between labels) collected as data by a detecting apparatus (e.g. a microscope). In certain embodiments, a labeling pattern is a sequence of “colors” in an order of their positions along a double-stranded DNA. In other embodiments a labeling pattern is a sequence of colors and distances between colors in an order of their positions along a double-stranded DNA.

A “distinct labeling pattern” or “distinctly labeled”, as used herein, refers to a labeling pattern of a region of a labeled double-stranded nucleic acid that is different from all other regions of nucleic acids in the genomic sample of interest and identifies the region relative to other regions in the sample. A certain level of complexity is required in a distinct labeling pattern depending on the length of the region that needs to be uniquely identified out of the total number of regions in the sample.

The term “reference pattern”, as used herein, refers to a labeling pattern derived from actual experiments or in silico, by taking part or all assay parameters into account. In certain cases, the reference genome is the same species as that of the genomic sample of interest.

### Description of Exemplary Embodiments

A method of genome analysis is provided. In certain embodiments, the method comprises: a) contacting a genomic sample comprising a double-stranded DNA with a site-specific nicking endonuclease to provide a nicked double-stranded DNA comprising a plurality of nick sites, in which the nicking endonuclease nicks a site adjacent to a variable nucleotide; b) contacting the nicked double-stranded DNA with a polymerase in the presence of a nucleotide composition comprising a first labeled nucleotide comprising a first label, thereby producing a labeled double-stranded DNA that is not labeled at every nick site; c) stretching out the labeled double-stranded DNA to provide a stretched, labeled double-stranded DNA; and d) imaging the stretched, labeled double-stranded DNA to identify a labeling pattern on the stretched labeled double-stranded DNA.

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method of Genome Analysis

A method of genome analysis is provided. In certain embodiments, the method comprises: a) contacting a genomic sample comprising a double-stranded DNA with a site-specific nicking endonuclease to provide a nicked double-stranded DNA comprising a plurality of nick sites that are adjacent to a variable nucleotide; b) contacting the nicked double-stranded DNA with a polymerase in the presence of a nucleotide composition comprising a first labeled nucleotide comprising a first label, thereby producing a labeled double-stranded DNA in which not every nick site is labeled by the first label; c) stretching out the labeled double-stranded DNA to provide a stretched, labeled double-stranded DNA; and d) imaging the stretched, labeled double-stranded DNA to identify a labeling pattern on the stretched labeled double-stranded DNA.

The nucleotide composition used in the method provides for labeling of some but not all of the nick sites. In certain cases, the nucleotide composition may contain only chain terminator nucleotides (e.g. one, two, three or all of the adenine-, guanine-, cytosine-, thymine-derived nucleotides, in which each of the nucleotides is distinguishably labeled). The nucleotide composition may also contain a combination of labeled and unlabeled nucleotides. In many embodiments described herein, the nucleotide composition contains only chain terminator nucleotides. Although a nucleotide composition may contain only chain terminator nucleotides (e.g. dideoxynucleotides), or only non-chain terminating nucleotides (e.g. deoxynucleotides), a combination of chain terminators and non-chain terminators are also envisioned.

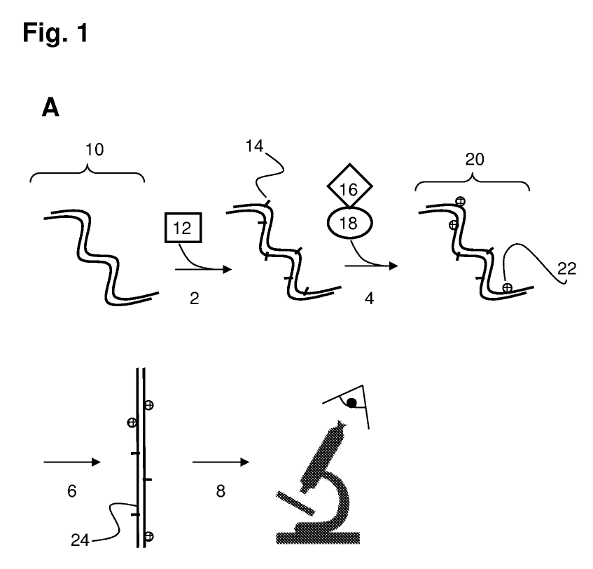

One embodiment chosen to illustrate the subject method is shown in FIG. 1 and is described in greater detail below. With reference to FIG. 1, the method may involve contacting 2 a genomic sample comprising double-stranded DNA 10 with site-specific nicking endonuclease 12 under conditions suitable for the site-specific nicking endonuclease to nick the backbone (i.e. hydrolyzes a phosphodiester bond in the DNA backbone) to produce a plurality of nick sites (e.g. 14) at different positions on the double-stranded DNA. Since the nicking endonuclease is site-specific, nick 14 is located within or adjacent to the recognition sequence of the site-specific nicking endonuclease. The nicked double-stranded DNA is then contacted 4 with polymerase 16 in the presence of nucleotide composition 18 comprising labeled nucleotide 22. The polymerase 16 then incorporates labeled nucleotide 22 into the double-stranded DNA in step 4. As a result, the double-stranded DNA becomes labeled in a site-specific manner. The labeled double-stranded DNA 20 is then stretched 6 so that the double-stranded DNA is elongated to remove tertiary structures. The labels (e.g., 22) on the stretched labeled double-stranded DNA 24 are then imaged 8 for analysis.

As shown in FIG. 1, the contacting step 2 may be performed by contacting a genomic sample comprising double-stranded DNA 10 with site-specific nicking endonuclease 12. In certain cases, the double-stranded DNA in the genomic sample have been fragmented by sonication or nebulization (e.g. to a size of about 10 kb to about 1000 kb or more), amplified, or partially purified prior to the contacting step 2. The double-stranded DNA 10 may also be treated with a ligase prior to contacting step 2 to avoid spurious labeling of sites not specifically nicked by the site-specific nicking endonuclease 12. The way and order of contacting the genomic sample with the site-specific nicking endonuclease may vary depending on the assay conditions. In certain cases, the site-specific nicking endonuclease may be added to a sample comprising the test genome. In other cases, the sample comprising the test genome may be added to a solution containing the site-specific nicking endonuclease. In certain cases, contacting steps 2 and 4 may be performed simultaneously so that the genomic sample comprising the double-stranded DNA is contacted with the site-specific nicking endonuclease, the polymerase, and the nucleotide composition all in the same time. Conditions and reagents suitable for the nicking activity of site-specific nicking endonuclease are known to one of skilled in the art. Exemplary methods and experimental conditions suitable for an active site-specific nicking endonuclease may be found in Jo K et al. (2007) PNAS 104:2673-2678 and Xiao M et al. (2007) Nucleic Acids Res. 35:e16.

As noted above, the site-specific nicking endonuclease employed in contacting step 2 is site-specific. In other words, the site-specific nicking endonuclease nicks the backbone of a double-stranded DNA in a sequence specific manner. The recognition sequence varies from one to the other and some site-specific nicking endonucleases along with their features are summarized in Table 1 below.

TABLE 1
Nicking endonucleases (recognition sequences are presented 5′-- 3′.)

| | | | Nucleotide 5′ | Nucleotide 3′ | | |
|---|---|---|---|---|---|---|
| | | Nick in top | to nick (for | to nick (for | Frequency in | Sites in |
| Nicking | Recognition | or bottom | proofreading | nick translation | random | Lambda |
| endonuclease | sequence | strand | labeling) | labeling) | sequence | genome |
| Nb.BbvCI | CCTCAvGC | Bottom | C | T | 1/16384 | 7 |
| Nb.BsmI | GAATGvC | Bottom | G | C | 1/4096 | 46 |
| Nb.BsrDI | GCAATGv | Bottom | X | C | 1/4096 | 44 |
| Nb.BtsI | GCAGTGv | Bottom | X | C | 1/4096 | 34 |
| Nt.A1wI | GGATCNNNN\hat{ } | Top | X | X | 1/1024 | 58 |
| Nt.BbvCI | CC\hat{ }TCAGC | Top | C | T | 1/16384 | 7 |
| Nt.BspQI | GCTCTTCN\hat{ } | Top | X | X | 1/16384 | 10 |
| Nt.BstNBI | GAGTCNNNN\hat{ } | Top | X | X | 1/1024 | 61 |

In the table above, the “v” or “̂” within each recognition sequence represents the location of the nick site for the corresponding site-specific nicking endonuclease relative to the recognition sequence. “v” denotes a nick site on the strand opposite of the recognition sequence, while “̂” denotes a nick site on the same strand of the recognition sequence. Also listed in this table are nucleotides immediately 5′ and 3′ to the nick site for each corresponding site-specific nicking endonuclease, in which the variable nucleotides are represented by “X” in the columns “nucleotide 5′ to nick” and “nucleotide 3′ to nick”. As seen in the table above, the nick site created by each site-specific nicking endonuclease may or may not be flanked by a variable nucleotide. In certain embodiments, there is at least one variable nucleotide adjacent to a nick site (e.g. two variable nucleotides flanking a nick site). In other embodiments, there is no variable nucleotide adjacent to the nick site at all.

One site-specific nicking endonuclease that does not have any variable nucleotide adjacent to its nick site is Nt.BbvCI. Nt.BbvCI recognizes the nucleotide sequence of CCTCAGC and nicks the backbone between cytosine (C) and thymine (T). Since C and T are known nucleotides that are part of the recognition sequence, there is no variable nucleotide adjacent to the nick site of Nt.BbvCI. In many embodiments, site-specific nicking endonucleases including Nt.BbvCI, Nb.BsmI, and Nt.BbvCI, are not used in the subject method because they do not nick adjacent to a variable nucleotide.

Nt.AlwI, on the other hand, nicks a site that is flanked by variable nucleotides on both sides. Nt.AlwI recognizes GGATCNNNN and nicks the backbone after four nucleotides 3′ to the C. The nick site of Nt.AlwI falls between two nucleotides, both of which may vary among different nucleic acid samples. As such, the nick site of Nt.AlwI is adjacent to or between two variable nucleotides. In other cases, the site-specific nicking endonuclease nicks a site adjacent to one variable nucleotide. One such site-specific nicking endonuclease is Nb.BsrDI, that recognizes the nucleotide sequence of GCAATG and nicks the opposite strand, as indicated in the table. As such, the nick site of Nb.BsrDI is between the nucleotide complementary to the last G in the recognition sequence (C) and a variable nucleotide.

As noted above, the subject method employs a site-specific nicking endonuclease that nicks a site adjacent to at least a variable nucleotide (e.g. a site flanked by two variable nucleotides). Examples of site-specific nicking endonuclease that may be used in the contacting step 2, as illustrated in FIG. 1, include but are not limited to Nb.BsrDI, Nb.BtsI, Nt.AlwI, Nt.BspQ1, and Nt.BstNB1. Other site-specific nicking endonuclease may be used as long as the nick site is adjacent is at least one variable nucleotide.

In certain embodiments, the method may employ more than one site-specific nicking endonuclease, e.g. two, three, or more different types of site-specific nicking endonuclease, in the contacting step 2. Where more than one site-specific nicking endonuclease is used to nick a double-stranded DNA of a genomic sample, at least one of the site-specific nicking endonucleases nicks a site adjacent to a variable nucleotide. Used in combination with the site-specific nicking endonuclease that nicks a site adjacent to a variable nucleotide, the additional one or more site-specific nicking endonucleases may or may not nick a site adjacent to a variable nucleotide. Any of the site-specific nicking endonuclease listed in Table 1 may be employed as the additional site-specific nicking endonuclease to be used in combination with the site-specific nicking endonuclease that nicks a site adjacent to a variable nucleotide.

Since many of the recognition sequences of the site-specific nicking endonuclease shown in Table 1 are common nucleotide sequences found in genomic DNA, the double-stranded DNA of the genomic sample under study may comprise a plurality of nick sites after the contacting step 2. Depending on the type of double-stranded DNA under study, there may be more than 1 (e.g., more than 2, more than 5, more than 10, more than 30, more than 50, more than 60, up to 100 or more) nick sites over any contiguous sequence of about 40,000 nucleotides. When there are too many recognition sequences of the site-specific nicking endonuclease used in the contacting step 2, resulting in a high density of nick sites along the double-stranded DNA, it may be desirable to prevent all of the nick sites from being labeled. Certain features of the subject method are available to decrease the amount of labeled sites relative to the total amount of nick sites and these features are discussed later below.

As noted above, the nicked double-stranded DNA produced by contacting step 2 is then labeled with polymerase 16 in the presence of nucleotide composition 18 comprising labeled nucleotides. This subject method provides several features in the contacting step 4 in order to generate labeling pattern of interest for subsequent visualization. The features may involve modifying the nucleotide composition and/or choosing the appropriate polymerase. Exemplary embodiments are presented below to further illustrate how the types of nucleotide composition and of the polymerase may be chosen to accommodate the various needs.

In certain cases, the nucleotide composition may allow for multi-color labeling, in which there may be at least two, three, or four distinguishably labeled nucleotides. For example, guanine-derived nucleotides have a detectable label that is different from adenine-, cytosine-, or thymine-derived nucleotides. Each type of labeled nucleotide is distinguishably labeled in the composition for multi-color labeling. In order to better describe a nucleotide composition comprising distinguishably labeled nucleotides, a labeled nucleotide in a nucleotide composition may be designated as a first nucleotide comprising a first label. In other embodiments, the nucleotide composition comprises an additional nucleotide to the first nucleotide that is different from the first nucleotide. This additional nucleotide type may be designated as a second nucleotide comprising a second label. In an alternative embodiment, the nucleotide composition may comprise a first labeled nucleotide and a second labeled nucleotide as described as well as a third nucleotide comprising a third label. In certain cases, the nucleotide composition may comprise all four nucleotides, each comprising a different label. As an example of a nucleotide composition comprising all four nucleotides, adenine may be considered to be a first nucleotide comprising red as the first label, guanine a second nucleotide comprising green as the second label, cytosine a third nucleotide comprising blue as the third label, and thymine a fourth nucleotide comprising yellow as the fourth label. In any nucleotide composition described herein, the composition may comprise a) only a first, b) only a first and a second, c) only a first, a second, and a third, or d) all four labeled nucleotides, but not any other nucleotides that can be a substrate for the polymerase used in the subject method. For nucleotide composition comprising chain terminators, non-chain terminators, or combination thereof as noted above, the designation of first, second, third and fourth for nucleotides and their labels is not meant for the purpose of assigning a sequential order but rather to differentiate one nucleotide that is distinguishably labeled from another.

As described above, the detectable label of a nucleotide may comprise a tag that emits a color or a non-fluorescent tag that is further processed for visualization. “Color”, as used herein, refers to the wavelengths of a detectable label at which the maximum of the emission spectrum resides. For example, nucleotides labeled green have a maximum emission peak at about 510 nm.

In a related embodiment, the labeled nucleotides may be chain terminators. Labeled chain terminators can be used to incorporate a single site-specific label and block further extension. In an exemplary nucleotide composition in which there are a first and a second labeled chain terminator nucleotides, the first and second labeled chain terminator nucleotides may be adenine derivatives and guanine derivatives, respectively. The adenine-derived chain terminators may be labeled red as so red is a first label, while the guanine derived chain terminators may be labeled green so green is the second label. In another example, four-color labeling may employ first, second, third and fourth labeled chain terminators derived from A, G, C, and T, respectively, in which each of the first, second, third, and fourth labels emits a color different from each other.

In a related embodiment, the nucleotide mixture may comprise phosphorothioated nucleotides, e.g., nucleoside alpha-thiotriphosphates (also known as alpha-thionucleoside triphosphates). An exemplary nucleoside may be alpha-thiotriphosphates is 2′-deoxyadenosine 5′-O-(1-thiotriphosphate). Nucleoside alpha-thiotriphosphates can be incorporated by various DNA polymerases, including T4 DNA polymerase (Romanuik and Eckstein, (1982) J. Biol. Chem. 257: 7684-7688), Taq polymerase, and 9N DNA polymerase (Yang et al., (2007) Nucl. Acids. Res. 35: 3118-3127). Nucleoside alpha-thiotriphosphates can be used to protect DNA from exonuclease degradation (Yang et al., (2007) Nucl. Acids. Res. 35: 3118-3127). In embodiments, nucleotide mixtures comprising nucleoside alpha-thiotriphosphates are used to inhibit further degradation by the 3′ to 5′ exonuclease activity of a proofreading polymerase. For example, a polymerase with a proofreading exonuclease activity may digest the native base 5′ to a nick, and incorporate a labeled, chain terminator, nucleoside alpha-thiotriphosphate in place of the original base. Thus the incorporated base may be resistant to further digestion by the exonuclease activity. The newly incorporated base would serve 3 functions: it would fluorescently label the nick site (with a label corresponding to the identity of the base 5′ to the nick); it would stop further nucleotide incorporation, allowing specificity of labeling (from the chain terminator modification); and it would protect the labeled site from further degradation by the proofreading exonuclease activity (from the phosphorothioate linkage).

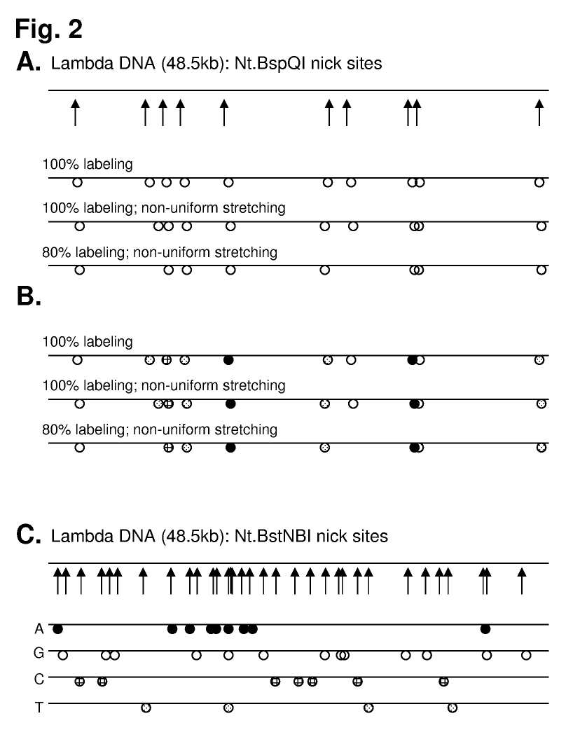

Certain aspects of multi-color labeling are illustrated in the comparison between FIGS. 2A and 2B. Shown in the figure are Nt.BspQI nick sites on the lambda DNA, pointed out by the arrows. The site-specific nicking endonuclease Nt.BspQ1 creates a nick on one strand of a double-stranded DNA near the sequence GCTCTTC, which occurs roughly once every 16384 bp in a random sequence. In the 48,502 bp lambda genome, there are ten occurrences of this recognition sequence. The illustrations immediately below show labeled chain terminators incorporated in the nick sites along the stretched DNA. FIG. 2A depicts a single-color labeling method in which all nucleotides (i.e. first, second, third, and/or fourth nucleotides) are labeled with a single-color. As such, the nucleotides are not distinguishably labeled and all labeled nick sites are represented by open circles in FIG. 2A. In contrast, a four-color labeling embodiment of the subject method is depicted in FIG. 2B in which a nucleotide composition comprising all four distinguishably labeled nucleotides is used. Nick sites labeled with adenine-derived labeled chain terminator are represented as filled circles, those with guanine-derivatives open circles, those with cytosine-derivatives criss-cross, and those with thymine-derivatives dotted. Single-color labeling (FIG. 2A) and four-color labeling (FIG. 2B) are compared under conditions affected by labeling efficiency and non-uniformity of stretching. The labeled nick sites are presented as circles along the length of the stretched lambda DNA in three patterns, each representing a labeling pattern under one of three conditions: 100% labeling of nick sites, 100% labeling of nick sites but non-uniform stretching of the DNA, or 80% labeling of nick sites in combination with non-uniform stretching of the DNA. As seen in the figure, when all labeled nucleotides have the same color label and the nick sites are labeled with a single-color, a specific pattern of label sites separated by predicted distances is created. However, if labeling is incomplete, or if the DNA stretching is variable, the label and distance information are compromised, resulting in a degraded label pattern.

Accordingly, to avoid producing a degraded labeling pattern, the subject method does not use nucleotide compositions in which the nucleotides are not distinguishably labeled. However, if the nicked double-stranded DNA is contacted with a polymerase in the presence of a mixture of four distinguishably-labeled, chain terminator dNTPs (e.g., ddA-PA-5dR6G, ddC-EO-5dTMR, ddG-EO-5dR110, ddT-EO-6dROX), as shown in FIG. 2B, a multi-colored coordinate system is produced. Each colored spot would contain a single label. The multiple colors create an information-rich label pattern. The multi-color labeling pattern may be robust to problems such as incomplete labeling or differential DNA stretching.

In addition to using more than one color label in the nucleotide composition, the nucleotide composition may also be free of one or more types of labeled nucleotides to control for a desired amount of labeling (e.g. have only a first, only a first and a second, or only a first, a second, and third labeled nucleotides). In certain embodiments, the number of labeled nick sites is less than the total number of nick sites. As noted previously, the ability to decrease the amount of labeling relative to the amount of nick sites may provide improvement in image resolution because a plurality of nick sites may be present at too high of a density for resolution by visible light. In some cases, the density of labeled nucleotides incorporated into a region of a double-stranded DNA may be no more than about once every 1000 bp, 2000 bp, 5 kb, or 10 kb, such that the distance between labels is resolvable by a light microscope. In certain cases, the distance between labels is at least near or above the diffraction limit for visible wavelengths of light.

The nucleotide sequences of the genome under analysis may be analyzed to identify the number of A, T, C, and G present at the variable nucleotide position. An appropriate nucleotide composition can then be designed to achieve the desired labeling density. A nucleotide composition free of at least one type of labeled nucleotide allows for labeling only a proportion of nick sites. Examples are presented below for the subject method employing labeled chain terminator nucleotides. If the nucleotide composition comprises only a first labeled chain terminators, then only about 10-40% of nick sites would be labeled. If the nucleotide composition comprises only a first and a second labeled chain terminator, then only about 30-70% of nick sites would be labeled. If the nucleotide composition comprises only a first, a second, and a third labeled chain terminators, then only about 50-85% of nick sites would be labeled. Finally, if the nucleotide composition employed comprises all of a first, a second, a third, and a fourth labeled chain terminators, then 100% of the nick sites would be labeled. As such, assuming all nucleotides are present at roughly an equal frequency at the variable nucleotide position, even with 100% labeling efficiency, having a nucleotide composition free of one or more types of labeled chain terminator would leave about 25% or more of the nick sites unlabeled. For example, a nucleotide composition without labeled adenines, for example, may leave nick sites adjacent to adenines unlabeled. Consequently, the number of labeled nick sites would be less than the total number of nick sites.

In a circumstance where A, T, C, and G nucleotides are not present at equal frequency in the double-stranded DNA to be labeled, the choice of nucleotide to be included in the nucleotide composition may be based on the region of the genome where the nick sites are located and the frequency for each nucleotide in that region. For example, depending on the nature of the analysis, a lower labeling density may be desirable for one region of the genome but not another.

Several embodiments of the subject method in which there is only one type of labeled nucleotides in the nucleotide composition are shown in FIG. 2C. FIG. 2C illustrates a segment of the lambda DNA with arrows pointing at Nb.BstNBI nick sites. Below the segment of lambda DNA showing Nb.BstNBI nick sites are four schematics showing the nick sites where each of the four corresponding types of labeled chain terminators would be incorporated along the segment of the lambda DNA. Nick sites labeled with adenine-derived labeled chain terminator are represented as filled circles, those with guanine-derivatives open, those with cytosine derivatives criss-cross, and those with thymine-derivatives dotted. The segment of lambda DNA shown has at least 35 nick sites after contacting with Nt.BstNBI and due to the proximity of several nick sites, resolution in certain regions may prove to be difficult using a light microscope. However, as seen in the schematics below, labeling in the presence of a nucleotide composition with only one type of labeled nucleotides greatly reduces the number of incorporated labels compared to the total number of the plurality of nick sites. Consequently, the density of incorporated labels also decreases in many cases so the individual labels may be resolved by the subsequent imaging step. For example, if only thymine-derived labeled chain terminators are used in the nucleotide composition, only 4 nick sites out of the at least 35 nick sites would be labeled in the segment of lambda DNA shown. The incorporated 4 labels would also be easily resolved because they are spaced far apart from each other. Accordingly, the nucleotide composition may comprise a) only a first, b) only a first and a second, or c) only a first, a second, and a third labeled nucleotides in order to decrease the number of incorporated labels relative to the total number of nick sites.

In certain embodiments, a nick translation polymerase is used for contacting step 4 and it incorporates a labeled nucleotide 3′ to the nick site. In the presence of nucleotides, a nick translation polymerase moves in the 5′ to 3′ direction from the nick site to displace and cleave one or more nucleotides from the 5′ end of the downstream DNA strand (3′ to the nick site), while simultaneously adding new nucleotides to the 3′ end of the upstream DNA strand. In this process, nucleotides are replaced (e.g., with dye-labeled analogs) and the nick continues to move in a 5′ to 3′ direction (unless chain terminators are added). DNA polymerases possessing strand displacement activity, but lacking 5′ nuclease activity, can also be used to add nucleotides to the 3′ end of the upstream DNA strand (5′ to nick). In certain cases, a proofreading polymerase is employed to incorporate labeled nucleotides. In such embodiments, a proofreading polymerase may move in the 3′ to 5′ direction to remove one or more nucleotides from the 3′ end of a DNA strand if the 3′ terminal nucleotide is a mismatch, but may also occur under conditions where exonuclease activity is favored over polymerization. Exemplary conditions in which a proofreading polymerase may move in the 3′ to 5′ direction: in the absence of nucleotides, in the absence of the correct next nucleotide (and low concentrations of incorrect nucleotides), or using a combination of polymerase, nucleotide analog(s), and reaction conditions that favor excision and replacement of the 3′ terminal nucleotide with a complementary labeled chain terminator over misinsertion of a non-complementary labeled chain terminator.

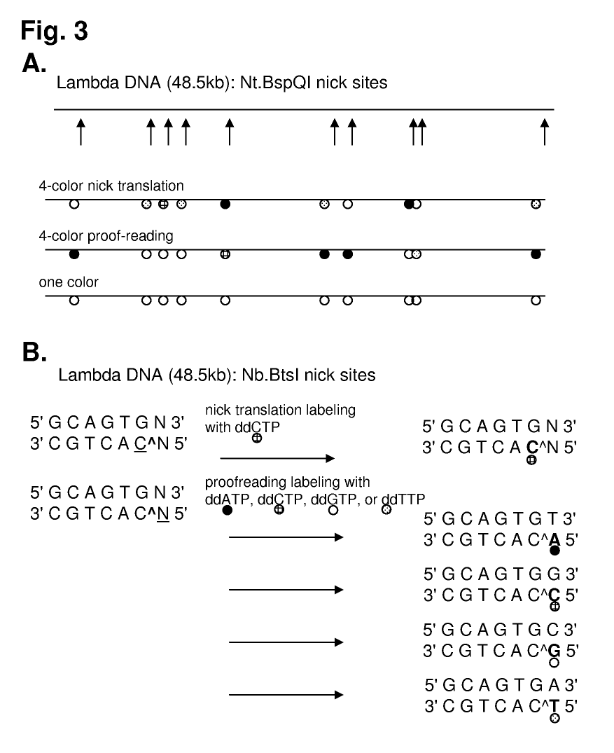

Either a nick translation polymerase or a proofreading polymerase may be used in the presence of a nucleotide composition that allows for four-color labeling described above. FIG. 3A illustrates 4-color labeling patterns of lambda DNA nicked with Nt.BspQI using either a nick translation polymerase or a proofreading polymerase. As apparent from this figure, 4-color labeling produces an information-rich pattern compared to one-color labeling. Furthermore, when one-color labeling is used, the pattern does not change whether a nick translation polymerase or a proofreading polymerase is used. FIG. 3A further shows that the pattern resulted from the use of a nick translation polymerase is different from that resulted from the use of a proofreading polymerase since different nucleotides are incorporated. Hence, the choice between the two types of polymerase would allow for generation of different labeling patterns when more than one color is used in the nucleotide composition.

Depending on the site-specific nicking endonuclease used in contacting step 2, a nick translation or a proofreading polymerase may incorporate a labeled nucleotide into the nicked double-stranded DNA to replace a known nucleotide in the recognition sequence or a variable nucleotide. If a nick translation polymerase is used in conjunction with site-specific nicking endonuclease that creates a nick with a variable nucleotide 3′ to the nick site (e.g. Nt.AlwI, Nt.BspQI, and Nt.BstNBI), nick translation polymerase would replace a variable nucleotide when incorporating a labeled nucleotide into the double-stranded DNA. Similarly, if a proofreading polymerase is used in conjunction with a site-specific nicking endonuclease that creates a nick with a variable nucleotide 5′ to the nick site, a variable nucleotide would be replaced. When a variable nucleotide is replaced during contacting step 4, nucleotide composition may be altered as described above to be free of one or more labeled nucleotide types. An appropriate polymerase may be chosen in combination with a certain nucleotide composition to reduce the number of labeled nick sites relative to the total number of nick sites as shown in FIG. 2C.

The nucleotide sequences of the genome under analysis may be analyzed to identify the number of A, T, C, and G present at the variable nucleotide position. Assuming the percentage of all four nucleotides, A, T, C, and G, in the nucleotide sequence of the double-stranded DNA are about equal, the probability that the variable nucleotide is any of the four nucleotides is roughly 25%. Hence, if a site-specific nicking endonuclease used in contacting step 2 creates a nick site with a variable nucleotide 5′ to the nick site, a proofreading polymerase would label an estimated 25% of nick sites in the presence of a nucleotide composition with only a first labeled nucleotide. If the nucleotide composition comprises a first and a second labeled nucleotides, the percentage of nick sites that would be labeled is estimated to be 50%. When there are less than all four types of labeled nucleotides present for a double-stranded DNA nicked by such a site-specific nicking endonuclease and contacted with a proofreading polymerase, the number of labels incorporated may be less than the total number of nick sites. In a similar fashion, in embodiments where there is a variable nucleotide 3′ to the nick site, nick translation polymerase may be used in the presence of a nucleotide composition depleted of one or more types of labeled nucleotides. Descriptions are presented below to further illustrate how to label a number of sites less than the total number of nick sites when there is a variable nucleotide adjacent to the nick site to be replaced by the polymerase of choice.

The choice between using a nick translation and a proofreading polymerase may rest upon whether a variable nucleotide adjacent to the nick site would be replaced. If a site-specific nicking endonuclease is used in which there is not a variable nucleotide 3′ to the nick sites, nick translation polymerase would only incorporate the same known nucleotide at every nick sites. As a result, nick translation polymerase would label every nick sites on a double-stranded DNA. For example, in an embodiment where Nb.BsrDI is used as the site-specific nicking endonuclease, there is no variable nucleotide 3′ to the nick site, so only cytosine-derived nucleotides would be incorporated if a nick translation polymerase is used in conjunction with Nb.BsrDI. In such a scenario, a nick translation polymerase would label all the nick sites assuming 100% labeling efficiency. As a result, the density of labeling would be comparable to the density of the nick sites. In certain cases, labeling of every nick site may not be desirable due to labels in images that are difficult to resolve, especially if the recognition sequence happens to be present at a very high density along a double-stranded DNA of a genomic sample. However, if there is a variable nucleotide 5′ to the nick site (e.g. nick site created by Nb.BsrDI), a proofreading polymerase may be used in conjunction a modified nucleotide composition that is free of one or more types of labeled nucleotide to decrease the amount of labeling.

As such, in cases where a site-specific nicking endonuclease is used in which there is only a variable nucleotide 5′ to the nick site but not 3′, choosing a proofreading polymerase allows the incorporation of labels in a selected group of nick sites out of the plurality by modifying nucleotide composition. As shown in FIG. 3B, when the site-specific nicking endonuclease employed nicks a site where there is only a variable nucleotide 5′ but not 3′ to the nick site, a proofreading polymerase would allow a selected number of sites to be labeled by using a modified nucleotide composition. Similarly, in cases where a site-specific nicking endonuclease is used in which there is only a variable nucleotide 3′ to the nick site but not 5′, a nick translation polymerase may be chosen. If there are variable nucleotides on both sides of the nick sites, as shown in FIG. 3A, either types of polymerase may be employed depending on the type of labeling pattern to be generated.

Accordingly, the nucleotide composition comprising labeled nucleotides (e.g. chain terminators) used in step 4 may be adjusted not only to accommodate the type of site-specific nicking endonuclease and polymerase used but also the amount of labeling desired for the double-stranded DNA of the genomic sample. In embodiments where the recognition sequences of a site-specific nicking endonuclease is commonly found in the genomic sample so as to result in a double-stranded DNA comprising nick sites present in too high of a density that interferes with the imaging resolution, the amount of labeling at nick sites may be decreased in accordance with the subject method to enable adequate resolution for the subsequent imaging step 8.

Since the recognition sequences of site-specific nicking endonucleases are known together with a wide availability of genomic sequences of interest, the number and the types of labeled nucleotides incorporated into a nicked double-stranded DNA may be predicted based on the type of site-specific nicking endonuclease and polymerase employed in the subject method. Based on this available information, various strategies may be devised in the same vein as the exemplary embodiments presented above to choose a polymerase and a nucleotide composition suitable for the analysis of the genomic sample.

Referring to FIG. 1, contacting steps 2 and 4 may be carried out in vitro or in situ. Cell extracts and tissue preparing may be utilized in these contacting steps. All steps of an in vitro labeling method may also be performed in a single tube. In other cases, steps may be performed on a substrate. For example, the substrate genome may be immobilized onto a bead or a planar surface.

After the nicked double-stranded DNA are labeled with the labeled nucleotides, represented by 22, in FIG. 1, the labeled double-stranded DNA are stretched out 6 to provide a stretched labeled double-stranded DNA 24 and imaged 8 to identify a labeling pattern. Many ways for stretching nucleic acid including the stretching devices used therein are known in the art. In certain cases, the labeled genome is stretched out into a linear form in order to detect the labels on the double-stranded DNA. Double-stranded DNA in aqueous solutions usually assumes a random-coil conformation. Similar to the method used in Fiber-FISH, the labeled genome comprising coiled DNA molecules may be unwound and stretched into a linear form on a modified glass surface and individually imaged by light microscopy, e.g. confocal, epifluorescence, internal reflection fluorescence. Briefly, the method may involve the following steps. First, the double-stranded DNA is pipetted onto the edge of a glass slide. The solution comprising the double-stranded DNA is then drawn under the coverslip by capillary action, causing the double-stranded DNA molecules of the genome to be stretched and aligned on the coverslip surface. As a result, an array of combed single DNA molecules is prepared by stretching molecules attached by their extremities to a glass surface with a receding air-water meniscus. This method is also referred to as molecular combing. By detecting the labels on the combed double-stranded DNA, labels may be directly visualized, providing a means to construct physical maps and to detect micro-rearrangements. Details of a method using microscopy to detect stretched genomic DNA may be found in Xiao M et al. (2007) “Rapid DNA Mapping by fluorescent single molecule detection” Nucleic Acids Res. 35:e16.

In other embodiments, the DNA molecules of the genome may be stretched 6 as they flow through a microfluidic channel. The hydrodynamic forces in a microfluidic channel generated in laminar flow help to uncoil and to stretch the DNA molecules as they travel with the flow. The solution is pressure driven to provide a flow acceleration over a distance comparable to the size of the DNA molecule. In this approach, a stretched DNA molecule travels through posts of focused light to excite a fluorophore label, for example. The label is detected as the DNA molecules pass through the detectors placed appropriately to capture the signal emitting from the microchannel. Details of using microfluidic channel to stretch and analyze single molecules may be found in US Pat Pub 20080239304 and 20080213912, disclosures of the patent publications are incorporated herein by reference.

In alternative embodiments, the DNA molecules of the genome may be stretched as they flow through a nanofluidic channel. In these embodiments, the nanofluidic channel may have a diameter of less than 200 nm, for example, less than 150nm, less than 100nm, less than 50 nm, or less than 20 nm. The confinement of the DNA molecules in the nanochannels leads to elongation of the DNA molecules, allowing optical interrogation. See e.g., Tegenfeldt et al (2004) Proc. Nat. Acad. Sci. USA 101:10979-10983; and Douville et al. (2008) Anal. Bioanal. Chem. 391:2395-2409.

After the labeled double-stranded DNA is stretched out, the stretched labeled double-stranded DNA is imaged to identify a labeling pattern. As mentioned above, the stretched labeled double-stranded DNA may be imaged 8 by employing various embodiments of microscopy described above, or by scanning during or after the stretching step 6. The imaging of the stretched labeled double-stranded DNA allows detection of the labeled nucleotides on the stretched double-stranded DNA 24. If the label is fluorescent, the presence of the label may be detected by the human eye, a camera, flow cytometry, or scanning fluorescence detectors, or a spectrometer, etc. If the nucleotide label is a tag composed of synthetic compounds, nucleic acids, amino acids, or a combination of both nucleic acids and amino acids, prior to imaging step 8, the double-stranded DNA may be processed to visualize the tag via binding to an epitope presented on the tag, primer extensions, sequencing, or additional processing to identify and locate the label, for example.

The labeling pattern obtained from the imaging step 8 may then be analyzed by a human or a computer programmed to analyze or compare labeling patterns. The image provides information derived from the double-stranded DNA with labeled nucleotides incorporated. In some embodiments, the labeling pattern is analyzed by recording a sequential order of colors in order of their positions along a length of the double-stranded DNA. The distance between any pair of labels may also be recorded. This sequential order of colors and/or distances between colored labels conveyed by the code allows the genomic context to be identified for the region of interest. In certain cases, a pattern of fluorescent labels may be recorded in forms of images or tables correlating emission wavelengths over the length of the double-stranded DNA. As described below, the code representing the labeling pattern may also be presented as values of emission wavelength in order of position of labeled nick sites along the double-stranded DNA.

These data recorded as a code represents the region of the double-stranded DNA into which the labels are incorporated. If the data comprises only two colors (e.g. red (R) and green (G)), or two distances (e.g. long (L) and short (S)), the code is considered to be binary. In a binary format, if the code has 2 bits, there are 2²=4 unique codes. E.g., RR, GG, RG, and GR or LL, LS, SL, and SS. The code may have 10 bits, providing for 2¹⁰=1024 unique codes. Accordingly, depending on the number of colors and distances in the code, the number of discrete units of information in a code may be designed so that sufficiently long regions in a genome may be uniquely identified. For example, in a scenario where a genome of about 245 million base pairs is divided up into consistent regions of about 10 kb to 100 kb in length, each requiring a unique identifier, there would be about 2,450 to about 24,500 regions. Where the subject method employs a binary code system, a 12 to 15 bit-code allows for 4,096 to 32,768 unique identifiers. As such, a 12 to 15 bit-code may adequately cover the whole genome although bit-codes beyond 15 bits are also envisioned herein. The bit required may be different to accommodate other scenarios (e.g. where the genome may be divided up into regions of various sizes, resulting in different number of regions).

Where the code comprises more than 2 colors and/or distances between colors, the code is then higher in complexity than the binary code so the amount of information units required to generate the same number of unique identifiers would be lower. For example, if the code contains 3 colors, an 8 to 10 trit-code would provide 6,561 to 59,049 unique identifiers. If the code contains 4 colors or 2 colors and 2 distances, a 6 to 8 unit-code would provide 4,096 to 65,536 unique identifiers, etc. In light of what has been described, various coding systems may be designed accommodate the various means of labeling genomic DNA or vice versa.

In certain cases, the code may be compared to a database of reference codes from control reference genome that has been labeled in the same way as the genomic sample of interest, either experimentally or in silico. If the code is found to be the same as one that is identified by the reference, the region of double-stranded DNA under study is identified to be the same as that of the reference. For example, if the code is red, red, green, green, and cytoband q34 of human chromosome 9 is the only expected region in the human genome that also has the same labeling pattern, then the region of double-stranded DNA under study is confidently identified to be region q34 of chromosome 9. Distance between labels may also be incorporated into the code to increase the specificity of the code for each identified region.

As noted previously, the subject method involves the analysis of a double-stranded DNA in a genomic sample. The genomic DNA may undergo staining, shearing, fragmentations, purification, etc., prior to being contacted with the site-specific nicking endonuclease in the method. In certain embodiments, the double-stranded DNA contacted with the site-specific nicking endonuclease and later the polymerase is at least 10, 50, 100, 500, 1000 or more kb up to a whole intact chromosome in length. The labeling pattern generated by the subject method may be derived from a contiguous stretch of double-stranded DNA that is at least 10, 50, 100, 500, 1000 kb, up to a whole intact chromosome.

The site-specific nicking endonuclease that may be used in the subject method includes any nuclease the specifically nicks the backbone in a duplex DNA in sequence specific manner. In certain embodiments, the site-specific nicking endonuclease encompasses those presented in Table 1 and derivations thereof. The site-specific nicking endonuclease employed may be a variant that exists in nature or a recombinant variant. It would be apparent to one of skilled in the art the variants of site-specific nicking endonuclease that can be employed in the subject method based on numerous studies on endonucleases in the art, as illustrated in Jeltsch et al. Trends Biotechnol. 14:235-8, 1996. Many site-specific nicking endonucleases are known in the art and commercially available.

The site-specific nicking endonuclease may be of a bacterial restriction modification system, of a mammalian origin or a hybrid of various origins. Recognition sequences and protein sequences of exemplary bacterial or mammalian site-specific nicking endonuclease are known and deposited in databases such as the REBASE restriction enzyme database, or NCBI's GenBank database.

As noted above, in certain embodiments, the site-specific nicking endonuclease creates a nick on a strand of a double-stranded DNA in a sequence-specific manner. In certain cases, the recognition sequence may comprise 4, 5, 6, 8, up to 10 or more nucleotides or nucleotide pairs. For example as shown in Table 1, the recognition sequence of Nb.BbvCI comprises 7 nucleotides, all of which are determined while the recognition sequence of Nt.BstNBI comprises 9 nucleotides, four of which are undetermined and so can vary among different nucleic acid samples.

As discussed above, the nucleotide composition used in the subject methods may comprise a) only first labeled nucleotide, b) only first and second labeled nucleotides, or c) only first, second, and third labeled nucleotides labeled nucleotides. In certain cases, the composition may comprise all four types of labeled nucleotides (e.g. adenine-, cytosine-, guanine-, thymine-derived chain terminators). In alternative embodiments, the composition may also comprise only non-chain terminating nucleotides or a combination of non-chain terminating nucleotides and chain-terminators. Where there is more than one type of labeled nucleotides, each type is distinguishably labeled. The label comprises a detectable component that can be either directly visualized or be processed for indirect visualization. Detectable labels are known in the art and need not described in detail herein. Briefly, exemplary detectable components include radioactive isotopes, fluorophores, fluorescence quenchers, affinity tags, e.g. biotin, crosslinking agents, chromophores, colloidal gold particles, beads, quantum dots, etc. In certain embodiments, the detectable label, such as biotin, may require incubation with a recognition element, such as streptavidin, or with secondary antibodies to yield detectable signals. In other embodiments, the detectable label, such as a fluorophore, may be detected directly without performing additional steps.

Additional fluorescent dyes of interest include: xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc.(Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 TOPRO3 (Molecular Probes, Eugene, Oreg.). Further suitable distinguishable detectable labels may be found in Kricka et al. (Ann Clin Biochem. 39:114-29, 2002).

In certain cases, the double-stranded DNA under study is stained with a nonspecific label, such as an intercalating fluorescent dye or other dyes that would label DNA in a non-sequence specific manner (e.g. DAPI, Hoechst, YOYO-1, YO-PRO-1, or PicoGreen). In related embodiments, a labeled nick site may participate in fluorescence energy transfer (FRET) with an adjacent labeled nick site or with the stained DNA backbone. The FRET signal is then imaged the same way as the embodiments described above to generate a pattern of labeled nick sites in order of positions along the length of the stretched double-stranded DNA.

Where the nucleotide composition comprises chain terminators, the chain terminators may be of any nucleotide that may be incorporated into a double-stranded DNA by a polymerase but prevent subsequent removal or extension. Some exemplary chain terminators include dideoxynucleotides, phosphorothioated analogs, and acyclo-nitrogenous bases. Any other synthetic nucleotides that prevent further extension after being incorporated into a double-stranded DNA may be used as chain terminators in the subject method.

In addition to site-specific nicking endonuclease and the nucleotide composition, the method also involves the use of a polymerase. As described above, the polymerase employed may be a nick translation polymerase that moves in the 5′ to 3′ direction starting from a nick site or a proofreading polymerase that removes one or more nucleotides in the 3′ to 5′ direction starting from a nick site. In certain cases, the polymerase does not have strand displacement activity. The polymerase may not have processivity such that the polymerase cannot remove and incorporate nucleotides continuously. In certain embodiments, the polymerase removes and incorporates no more than 1, no more than 2, no more than 3, no more than 4, no more than 5, no more than 6, or up to no more than 7 or more consecutive nucleotides each time it binds to a double-stranded DNA containing a nick site. Any enzyme capable of incorporating naturally-occurring nucleotides, nucleotides base analogs, or combinations thereof into a polynucleotide may be utilized in accordance with the present disclosure. As examples without limitation, the enzyme can be a primer/DNA template dependent DNA polymerase. Non-limiting examples of DNA polymerases include E. coli DNA polymerase I, E. coli DNA polymerase I Large Fragment (Klenow fragment), phage T4 DNA polymerase, or phage T7 DNA polymerase. The polymerase can be a thermophilic polymerase such as Thermus aquaticus (Taq) DNA polymerase, Thermus flavus (Tfl) DNA polymerase, Thermus Thermophilus (Tth) Dna polymerase, Thermococcus aggregans (Tag) DNA polymerase, Thermococcus litoralis (Tli) DNA polymerase, Pyrococcus furiosus (Pfu) DNA polymerase, Vent™ DNA polymerase, or Bacillus stearothermophilus (Bst) DNA polymerase. Furthermore, any molecule capable of using a DNA or an RNA molecule as a template to synthesize another DNA or RNA molecule can be used in accordance with the present invention. (e.g. self-replicating RNA).

Primer/DNA template-dependent DNA polymerases incorporate nucleotide triphosphates into the growing polynucleotide chain according to the standard Watson and Crick base-pairing interactions (see for example; Johnson, Annual Review in Biochemistry, 62; 685-713 (1993), Goodman et al., Critical Review in Biochemistry and Molecular Biology, 28; 83-126 (1993) and Chamberlain and Ryan, The Enzymes, ed. Boyer, Academic Press, New York, (1982) pp 87-108). Some primer/DNA template dependent DNA polymerases and primer are capable of incorporating non-naturally occurring triphosphates into polynucleotide chains when the correct complementary nucleotide is present in the template sequence. For example, Klenow fragment are capable of incorporating the base analogue iso-guanosine opposite iso-cytidine residues in the template sequence (Switzer et al., Biochemistry 32; 10489-10496 (1993). Klenow fragment are also capable of incorporating the base analogue 2,4-diaminopyrimidine opposite xanthosine in a template sequence (Lutz et al., Nucleic Acids Research 24; 1308-1313 (1996)).

Additional exemplary polymerases include mutant versions of polymerases (either engineered or of natural origin) which display an altered ratio of polymerase and exonuclease activities, relative to their wild-type versions. For example, mutants displaying a higher exonuclease activity, relative to the polymerase activity, may be useful as proofreading polymerases, as they may remove the nucleotide 5′ to the nick site more efficiently than the wild type version. Some examples of these mutants include Y387N, Y387S, or G389A mutants of the B-type DNA polymerase from Thermococcus aggregans (Bohlke et al., Nucleic Acids Research 28; 3910-3917 (2000)), the 1417V mutant of T4 DNA polymerase (Reha-Krantz and Nonay, J. Biol. Chem. 269: 5635-5643 (1994)), and R2271, G229A, F230Y, F230S mutants of phi29 DNA polymerase (Truniger et al., EMBO J. 15: 3430-3441 (1996)). The skilled artisan will understand that many of the known polymerases are highly homologous, and that relevant mutations in a polymerase of interest may be identified through sequence alignment to a characterized mutant polymerase.

Furthermore, exemplary polymerases may include mixtures of wild-type and mutant polymerases, or mixtures of different mutant polymerases. For example, a polymerase mixture with enhanced exonuclease activity, relative to the wild-type polymerase, may be constructed from a wild type polymerase combined with a mutant polymerase that has wild-type exonuclease activity and lower polymerase activity. Thus, the ratio of enzymatic activities in the polymerase mixture may be tuned to the desired ratio of exonuclease and polymerase activity. This flexibility will enable the exonuclease activity to be balanced with the polymerase activity in the proofreading labeling embodiments described herein, such that only one nucleotide is added 5′ to the nick site.

In carrying out the analysis of the image of the labeled stretched double-stranded DNA, a reference pattern derived from a reference genome may be used. The reference sequence may also undergo the subject method so that it is labeled in the same way as the genomic sample under interest. In other embodiments, the reference pattern may be derived in silico based on the information available about the reference sequence, such as those stored in databases. A reference sequence may be a sequence derived from an identified source or from the same species as the genomic sample under study. The source may be known to be homozygous or heterozygous for a particular genomic locus of interest. In certain cases, the source may be wild-type for a genomic locus of interest. The source may contain an allelic variant of interest. In certain cases, the reference sequence may be known so that the specific nucleotide sequences implicated in a genomic feature of interest (e.g. single nucleotide polymorphism, restriction fragment length polymorphism, genetic mutations, etc.) are known. The pattern of labeling may be predicted based on sequence data and the recognition site of the site-specific nicking endonucleases used.

The present disclosure also provides a system for sample analysis comprising: a) reagents to perform the subject method comprising a site-specific nicking endonuclease that nicks sites adjacent to variable nucleotide, and a nucleotide composition comprising a labeled nucleotide; b) a stretching device; c) an imaging workstation; d) a computer for recording; and e) a computer-readable medium comprising a database of reference patterns. The system may comprise one or more site-specific endonucleases as certain embodiments described above. The nucleotide composition provided by the system may also comprise various combinations of nucleotides described for the subject method. In certain cases, the nucleotide composition is free of at least one type of labeled nucleotide. Exemplary combinations include a) first labeled nucleotide, b) first and second labeled nucleotides, or c) first, second, and third labeled nucleotides. The nucleotide composition may comprise non-labeled nucleotides in addition to any of the labeled nucleotide. The nucleotides include chain terminators and/or non-chain terminator nucleotides. The stretching device and imaging work station encompass any instrument employed for the various stretching and imaging means described previously.

The system may include a computer programmed to record and store labeling pattern on a stretched double-stranded DNA. The system may encompass a storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer on a local or remote network. Similarly, a database of reference pattern may also be provided in a computer readable medium in the subject system.

### Kits

Also provided by the present disclosure are kits for practicing the subject method, as described above. The subject kit contains a site-specific site specific nicking endonuclease, a polymerase, a nucleotide composition comprising a labeled nucleotide, and reagents for nicking a double-stranded DNA and incorporating nucleotides into the nick sites. The kit may further contain a reference genome or information relating to a reference genome.

In additional embodiments, the kit may further comprise additional types of site specific nicking endonucleases and polymerases. In an alternative embodiment, the kit further comprises a) first labeled nucleotide, b) first and second labeled nucleotides, or c) first, second, and third labeled nucleotides. Labeled nucleotides may also be provided in various color labels and may be chain terminating, non-chain terminating, or a combination thereof. Kit may additionally provide unlabeled nucleotides. Specific combinations of site specific nicking endonuclease, polymerase, a nucleotide composition may be designed using the kit in accordance with individual needs.

The kits may be identified by the type of site specific nicking endonuclease, the recognition sequence of the site specific nicking endonuclease, the reference genome. The kits may also be identified by the type of polymerase in the kit, e.g. nick translation, proofreading, or both. The kits may be further identified by the method of analyzing the labeling pattern obtained from imaging the labeled stretched double-stranded DNA.

In addition to above-mentioned components, the subject kit typically further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the instructions, the kits may also include one or more control analyte mixtures, e.g., two or more control analytes for use in testing the kit.

In addition to above-mentioned components, the subject kit may include software to perform comparison of the pattern to one or more reference patterns.

### Utility

The subject method finds use in a variety of applications, where such applications are generally nucleic acid detection applications in which the presence of a particular nucleotide sequence in a given sample is detected at least qualitatively, if not quantitatively. In general, the above-described method may be used in order to identify a region in a genome based on the generated labeling pattern.

Since contacting steps 2 and 4 are both sequence dependent, the presence or absence of labeling in specific locations on double-stranded DNA is informative of the sequence information in those locations. By comparing the pattern of the labeled double-stranded DNA to those of a reference sequence, the genomic context and the identity of the labeled double-stranded DNA may be determined.

As noted above, the method provides analysis on a single molecule level, using methods such as those involving microscopy or a microfluidic/nanofluidic channels. In particular embodiments, the double-stranded DNA regions of interest are subjected to DNA stretching or confinement elongation prior to the imaging step. The subject method may also comprise recording the imaged labeled pattern as a code comprising a sequence of colors and/or distance between colors. The color represents the fluorescence emission of the labeled nucleotides incorporated into the double-stranded DNA. This recorded code may be used to compare with reference codes to identify the genomic context and the identity of the labeled double-stranded DNA (e.g. chromosome 9, region q34). The genomic context that may be assigned to a labeled double-stranded DNA identifies a segment of the double-stranded DNA on a scale of about 50, 100, 500, up to 1000 kb or more. In certain embodiments, the comparison between the recorded code and the reference may also help determine if there are chromosomal rearrangements or other sequence differences relative to the reference. Sequence alterations that may be detected include translocations, inversions, tandem duplications, insertions, deletions, SNPs, and other sequence mutations.

Analysis carried out using the method may be applied on a genomic scale that involves shearing, fragmenting, amplifying, or processing the double-stranded genomic DNA in other ways prior to contacting the genomic sample with a site specific nicking endonuclease. Although genomic sample may be complex, the code generated by the labeling patterns may be designed to be unique for the region of double-stranded DNA under study. Many labeling patterns may be generated in accordance with the many embodiments of the method described above so as to provide unique codes for each of a plurality of genomic regions. As mentioned above, each genomic region identified may be on a scale of about 50, 100, 500, up to 1000 kb or more in length.

Other assays of interest which may be practiced using the subject method include: genotyping, scanning of known and unknown mutations, gene discovery assays, genomic structural mapping, differential gene expression analysis assays, nucleic acid sequencing assays, and the like.

The pattern measured through the use of the subject methods can also be compared to a set of several reference patterns with the purpose of identifying the closest one. This might represent comparison between sequences coming from variants of a region or of an entire genome. Identification of the pattern in a sample genome may be useful for a wide variety of investigations, such as identifying origin of a crop, identifying species of fish or other animals, identifying pathogens, or distinguishing between a finite number of known genotypes. For example, a certain pattern in a human genome may identify that one DNA region is translocated or inverted with respect to the reference genome. Analysis of genomic rearrangements is useful in research on certain cancers, for example (De Lellis et al., Ann. Oncol. 18 Supp6: vi173-178 (2007)).

In certain cases, the genomic sample under study may be derived from a sample tissue suspected of a disease or infection. Performing the subject method to analyze the genomic sample from such sample tissues would be useful for disease diagnosis and prognosis. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain cases, the recognition sequence of a site specific nicking endonuclease overlaps a site of single nucleotide polymorphism (SNP) in the test genome or reference sequence. In other cases, the variable nucleotide adjacent to the nick created by the site specific nicking endonuclease maybe an SNP site. Since the nucleotide sequences of hundreds of thousand of SNPs from humans, other mammals (e.g., mice), and a variety of different plants (e.g., corn, rice and soybean), are known (see, e.g., Riva et al 2004, A SNP-centric database for the investigation of the human genome BMC Bioinformatics 5:33; McCarthy et al 2000 The use of single-nucleotide polymorphism maps in pharmacogenomics Nat Biotechnology 18:505-8) and are available in public databases (e.g., NCBI's onlisite-specific nicking endonuclease dbSNP database, and the onlisite-specific nicking endonuclease database of the International HapMap Project; see also Teufel et al 2006 Current bioinformatics tools in genomic biomedical research Int. J. Mol. Med. 17:967-73), the labeling of genomic DNA using a site specific nicking endonuclease to identify an SNP would be well within the skill of one of skilled in the art. The SNP may be known prior to choosing the site specific nicking endonuclease based on the site specific nicking endonuclease recognition site or the nucleotides adjacent to the nick sites of site specific nicking endonuclease. In certain embodiments, individual SNPs may differ among genomic sample as to destroy certain site specific nicking endonuclease recognition sequences or to change the identity of the variable nucleotide adjacent to the nick sites relative to a human genome reference sequence, and other SNPs may create site specific nicking endonuclease recognition sequences. Therefore, individual DNA samples may have different labeling patterns than that of a reference after being subjected to the method provided herein.

The above described applications are merely representations of the numerous different applications for which the subject array and method of use are suited. In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of sample analysis comprising:
a) contacting a genomic sample comprising a double-stranded DNA with a site-specific nicking endonuclease to provide a nicked double-stranded DNA comprising a plurality of nick sites, wherein said nicking endonuclease nicks a site adjacent to a variable nucleotide;
b) contacting said nicked double-stranded DNA with a polymerase in the presence of a nucleotide composition comprising a first labeled chain terminator nucleotide comprising a first label, thereby producing a labeled double-stranded DNA in which not every nick site is labeled with said first label;
c) stretching out said labeled double-stranded DNA to provide a stretched, labeled double-stranded DNA; and
d) imaging said stretched, labeled double-stranded DNA to identify a labeling pattern on said stretched labeled double-stranded DNA.

2. The method of claim 1, wherein said nucleotide composition comprises said first labeled chain terminator nucleotide and no other labeled nucleotides.

3. The method of claim 1, further comprising recording said labeling pattern as a sequence of distances between labeled nick sites along said stretched, labeled double-stranded DNA.

4. The method of claim 1, wherein said nucleotide composition comprises at least:
a) said first labeled chain terminator nucleotide comprising a first label; and
b) a second labeled chain terminator nucleotide comprising a second label, wherein said first label and said second label emit distinguishable colors.

5. The method of claim 4, further comprising recording said labeling pattern as a sequence of colors of labeled nick sites along said stretched, double-stranded DNA, wherein said colors are emitted by labeled chain terminator nucleotides at said nick sites.

6. The method of claim 4, further comprising recoding said labeling pattern as a sequence of colors of labeled nick sites and distances between said labeled nick sites along said stretched, labeled double-stranded DNA, wherein said colors are emitted by labeled chain terminator nucleotides at said nick sites.

7. The method of claim 4, wherein said nucleotide composition comprises:
a) said first labeled chain terminator nucleotide comprising a first label;
b) said second labeled chain terminator nucleotide comprising a second label;
c) a third labeled chain terminator nucleotide comprising a third label; and
d) a fourth labeled chain terminator nucleotide comprising a fourth label, wherein said first label, said second label, said third label, and said fourth label emit different colors.

8. The method of claim 1, wherein said labeling pattern identifies said double-stranded DNA as being a specific genomic region.

9. The method of claim 1, wherein said method further comprises
e) comparing said labeling pattern to a reference pattern.

10. The method of claim 1, wherein said polymerase incorporates said labeled chain terminator nucleotide in a position 5′ to said nick site.

11. The method of claim 1, wherein said polymerase incorporate said labeled chain terminator in a position 3′ to said nick site.

12. The method of claim 1, wherein said polymerase in contacting step comprises a 3′ to 5′ exonuclease activity in addition to a polymerase activity.

13. The method of claim 1, wherein said genomic sample comprises double-stranded DNA is contacted with a ligase prior to said contacting step a).

14. The method of claim 1, further comprising labeling a backbone of said double-stranded DNA to produced a labeled DNA backbone prior to imaging step d).

15. The method of claim 1, wherein said polymerase comprises a mixture of a plurality of enzymes.

16. The method of claim 1, wherein said labeled chain terminator nucleotide is a phosphorothioated nucleotide analog.

17. The method of claim 1, wherein said labeled chain terminator nucleotide is an acyclo-nitrogenous base.

18. The method of claim 1, wherein said double-stranded DNA is at least about 50 kilobases long.

19. The method of claim 12, wherein said polymerase is engineered to have an enhanced exonuclease activity, relative to the polymerase activity.

20. A system for sample analysis comprising:
a) reagents for performing the method of claim 1, wherein said reagents comprise a site specific nicking endonuclease that nicks sites adjacent to variable nucleotide, and a nucleotide composition comprising a labeled chain terminator nucleotide;
b) a stretching device;
c) an imaging workstation;
d) a computer for recording;
e) a computer-readable medium comprising a database of reference patterns.
